(19) United States
(12) Patent Application Publication
(10) Pub. No.: US 2010/0049249 A1
(43) Pub. Date: Feb. 25, 2010

(54) Suture anchor extension

(75) Inventor: Giuseppe Lombardo, New Port Richey, FL (US)

Correspondence Address: GENE WARZECHA;LINVATEC CORPORATION, 11311 CONCEPT BOULEVARD, LARGO, FL, 33773, US

(73) Assignee: LINVATEC CORPORATION

(21) Appl. No.: 12/229,567

(22) Filed: Aug. 25, 2008

(51) Int. Cl. A61B 17/04; A61B 17/10

(52) U.S. Cl. 606/232; 606/139

(57) ABSTRACT

A suture anchor extension for converting a non-fully threaded base suture anchor to a fully threaded suture anchor. The extension comprises a hollow tubular sleeve adapted to be axially aligned with and attached to the base suture anchor. One end of the sleeve engages the non-circular drive member on the base suture anchor and the other end engages a driver for turning the extension and base anchor into tissue at the work site. Suture attached to the base anchor is directed through the interior of the sleeve and, during installation, through the cannulation of the driver.

### FIELD OF THE INVENTION

The invention relates to suture anchors for re-attaching soft tissue to bone or other tissue. More particularly, the invention relates to screw-in suture anchors and devices for enhancing the strength of the attachment of the suture anchors to bone or other tissue.

### DESCRIPTION OF THE PRIOR ART

Suture anchors are commonly used to attach soft tissue to bone or other tissue during either open or closed surgical procedures. Suture anchors come in a variety of configurations including threaded or screw-in anchors which may be directly screwed into bone at the surgical site or screwed into a pre-formed (and, optionally, tapped) hole made in the bone. Certain suture anchors may be inserted into a pre-formed bone hole and then deployed to actually engage the wall of the bone hole. Suture anchors also come in a push-in configuration in which the anchor may be pushed into a pre-formed hole. All suture anchors serve as an anchor point for suture or suture-like material to attach soft tissue to the anchor and hold it near the bone or other tissue during the healing process.

One prior art suture anchor comprises an elongated, somewhat tapered body having a threaded outer surface, a tapered point at its distal end and an axially aligned drive portion at its proximal end. The drive portion is non-circular (e.g., hexagonal) and is designed to be engaged by a driver having a complementarily shaped non-circular, axially aligned recess to engage the drive portion to rotate the anchor relative to its axis in order to position it at the surgical site. This type of anchor is turned into the bone sufficiently so that the drive portion is recessed under the surface of the bone to avoid any abrasion of the re-attached soft tissue by the anchor. An eyelet or other suture attachment point is formed in the suture anchor, often transversely through the drive portion. While this structure is suitable for use as a suture anchor in bone and in tissue other than bone, a suture anchor has recently been developed primarily for use in bone. This anchor includes a thread along the entire length of its outer surface. This type of so-called fully threaded structure enables the thread at the proximal end of the suture anchor to engage the cortical layer of bone and thereby achieve a greater pull-out force.

The manufacture of such fully threaded suture anchor is somewhat compromised in view of the need to provide some attachment mechanism for sutures to be retained by the suture anchor. Accordingly, it is an object of this invention to produce a suture anchor assembly combining the advantages of fully threaded prior art suture anchors with the advantages of eyelet-bearing suture anchors.

It is another object of this invention to provide a device which is easily adaptable to a variety of thread configurations so that a fully threaded suture anchor assembly may be constructed for a variety of applications.

It is another object of this invention to provide a device which can be easily assembled with a prior art non-fully threaded suture anchor in order to produce a fully threaded suture anchor.

### BRIEF DESCRIPTION OF THE DRAWINGS

FIG. 1 is a front perspective, expanded view of the preferred embodiment of a suture anchor assembly, including a base suture anchor, suture anchor extension and a driver, constructed in accordance with the principles of this invention.

FIG. 2 is a side elevation view of the components of FIG. 1 assembled together.

FIG. 3 is side elevation view, partially in cross-section, of FIG. 1 in an assembled configuration.

FIG. 4 is a front perspective view of the distal end of the driver shown in FIG. 1.

FIG. 5 is a cross-sectional view of FIG. 2 taken along the line 5-5.

FIG. 6 is a cross-sectional view of FIG. 2 taken along the line 6-6.

FIG. 7 is a front perspective view of an alternate embodiment of a suture anchor driver.

FIG. 8 is a side elevation view, partially in cross-section, of the driver of FIG. 7 assembled with a suture anchor of FIG. 1 and an alternate embodiment of a suture anchor extension.

### DESCRIPTION OF THE PREFERRED EMBODIMENT

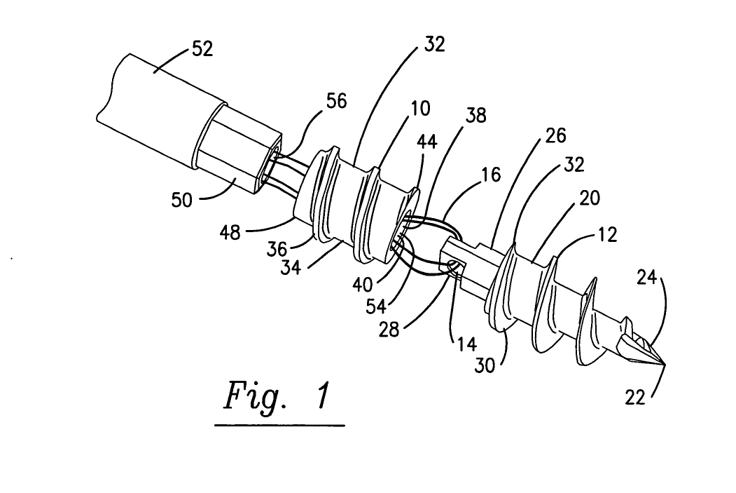

As shown in FIG. 1, the invention is a suture anchor extension 10 which is attachable to an existing eyelet-bearing suture anchor 12 to thereby create a fully threaded suture anchor. The suture anchor 12, sometimes herein called the base suture anchor, comprises an eyelet 14 through which one or more strands of suture 16 pass in a conventional manner. Anchor 12 comprises a body 20 having a point 22 at its distal end 24 and a non-circular driven member 26 at its proximal end 28. The outer surface of body 20 is provided with a thread 30 along its length although the proximal end of the thread 30 ends at a point 32 just distal to driven member 26. This enables driven member 26 to be engaged by the driving member of a driver during normal use of anchor 12 if it were to be used without the suture anchor extension.

Suture anchor extension 10 comprises a tubular body 32 having an outer cylindrical surface 34, a thread 36 extending along the entire length of the outer surface, and an inner surface 38 surrounding a hollow interior 40. Inner surface 38 is provided with a non-circular driving member 42 at the distal end 44 of extension 10 and a non-circular driven member 46 at proximal end 48. Driving member 42 is adapted to engage driven member 26 and driven member 46 is adapted to be engaged by non-circular driving member 50 formed at the distal end of cannulated driver 52. The axially aligned lumens 54 and 56 of extension 10 and driver 52, respectively, receive sutures 16 when the base anchor, extension and driver are assembled. The sutures pass through the cannulated driver and are secured to a cleat on the handle (not shown).

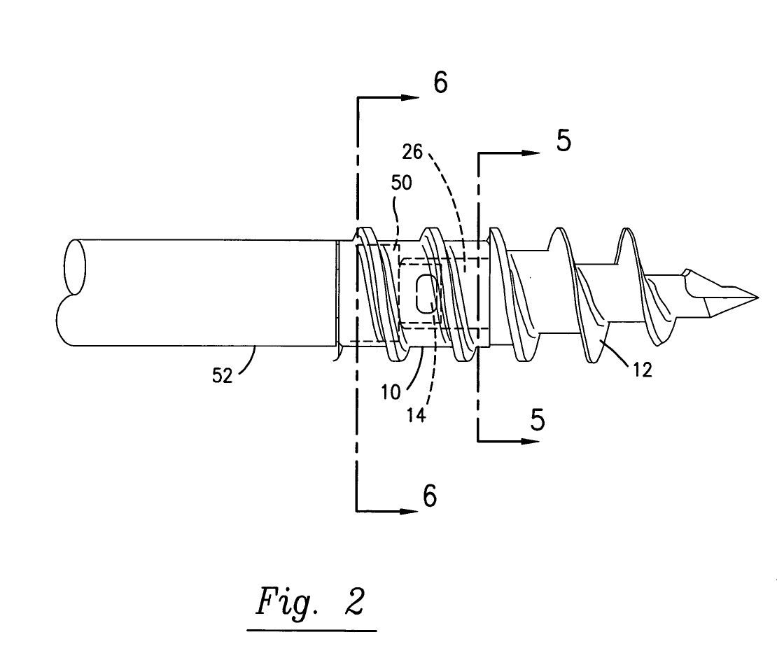

Suture anchor extension 10 may be made in a variety of sizes and shapes in order to mate with any selected non-fully threaded base suture anchor. The mating of the two pieces, i.e. the anchor and the extension, produces a new fully threaded suture anchor as best seen in FIG. 2. In a preferred embodiment the extension thread 36 is designed with the same pitch and size as the anchor thread 30. If the anchor assembly (i.e. extension 10 plus anchor 12) is inserted into a bone hole, the proximal end 48 of extension 10 will be flush with the bone surface (not shown). Also, the thread 36 will engage cortical bone lying near the bone surface. It will be understood that extension 10 and anchor 12 may be positioned rotationally about their common axes so that the threads 36 and 30 form essentially one continuous, unbroken thread. This may be accomplished by, for example, starting the distal end of thread 36 at a predetermined circumferential point relative to the hexagonal driving member 42 so that when drive members 42 and 26 become engaged the threads 36 and 30 will be automatically aligned. However, alignment is not essential.

The term “drive member” as used herein can refer to either a driving member or a driven member. It will be understood that a driving member is the one providing the force to rotate an element and a driven member is the one receiving the force. In the configuration of the preferred embodiment the extension 10 has a driven member 46 at its proximal end and a driving member 42 at its distal end.

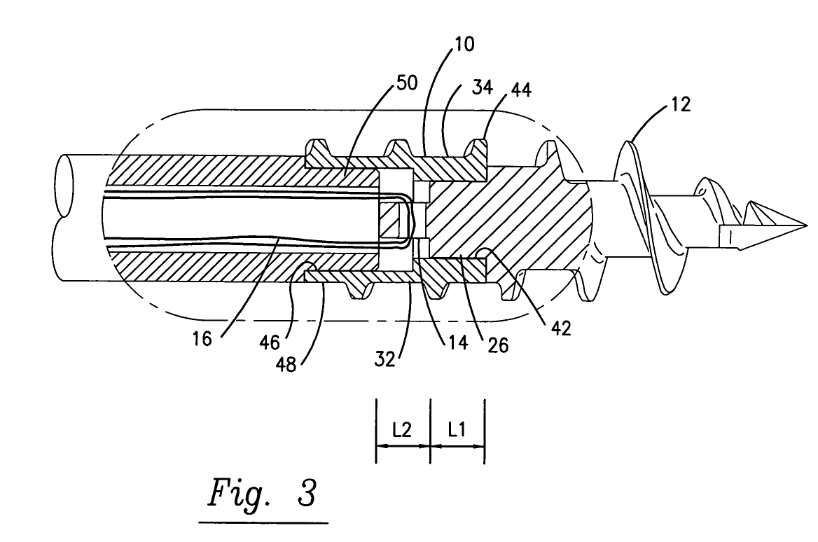

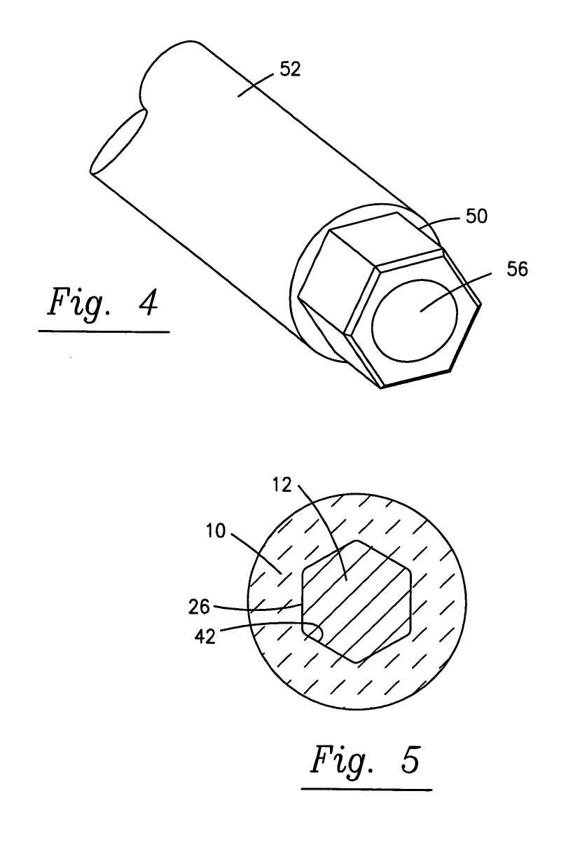

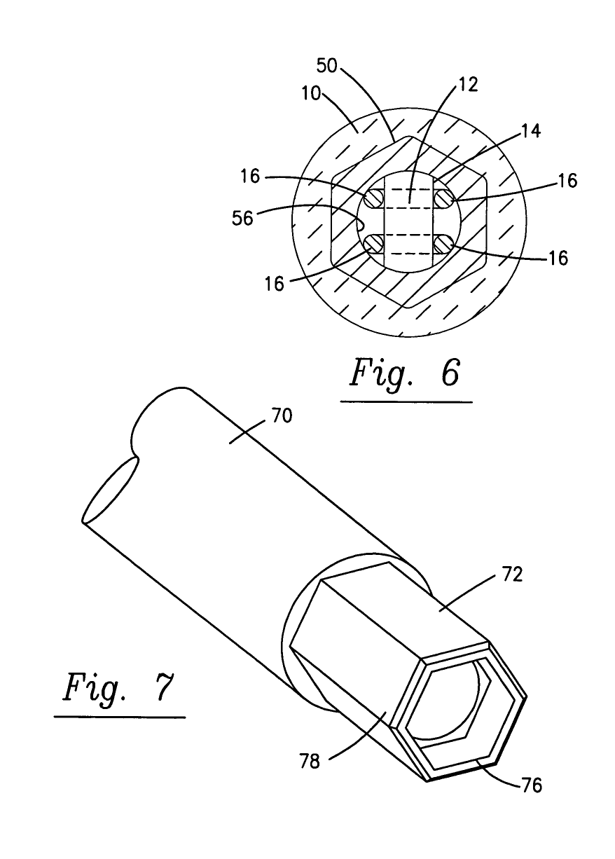

In the preferred embodiment, suture anchor extension 10 is axially aligned with and attached to or placed on anchor 12 after suture 16 is threaded through eyelet 14, as best seen in FIG. 2 (with suture 16 removed for clarity). This requires the suture to be drawn through the interior 40 of extension 10 and through the axial cannulations 56 of driver 52, as best seen in FIG. 3. Once the suture is so situated, the extension 10 may be placed on anchor 12 by engaging drive member 42 with drive member 26, and driver 52 may be placed on extension 10 by engaging drive member 50 with drive member 46.

Extension 10 may be attached to anchor 12 via a press- or friction-fit or may be bonded, welded, ultrasonically welded or otherwise secured. Alternatively, extension 10 may be loosely placed on anchor 12 since the interaction between the tissue being attached, sutures 16 and anchor 12 will tend to hold the extension and base anchor together. It will be understood that the extension and base anchor may both be made out of the same biocompatible material or of different materials. One may be bioabsorbable and the other not. One may be a polymeric composite material including bone growth inducting/conducting material and the other may be simply bioabsorbable or not. Any variety of combinations is possible.

The interior 40 of extension 10 may be formed in a variety of shapes to accommodate the anchor 12 and driver 50 with which the extension is intended to be used. In the embodiment shown in FIGS. 1-3, drive member 26 has a male hexagonal drive portion with an axial length L1, and an eyelet portion with an axial length L2. Consequently, to engage male drive member 26, female drive member 42 preferably has a hexagonal profile with an axial length equal to or greater than L1. The male drive member 50 is adapted to fit into female drive member 46 and abut the proximal end of drive member 26.

In the preferred embodiment the outer diameter of extension 10 may be uniform along its length, and approximately equal to the greatest outer diameter of anchor 12, or may be tapered outwardly from distal end 44 to proximal end 48. The proximal and distal ends of extension 10 preferably abut the driver and base anchor, respectively, to facilitate installation and use of the extension.

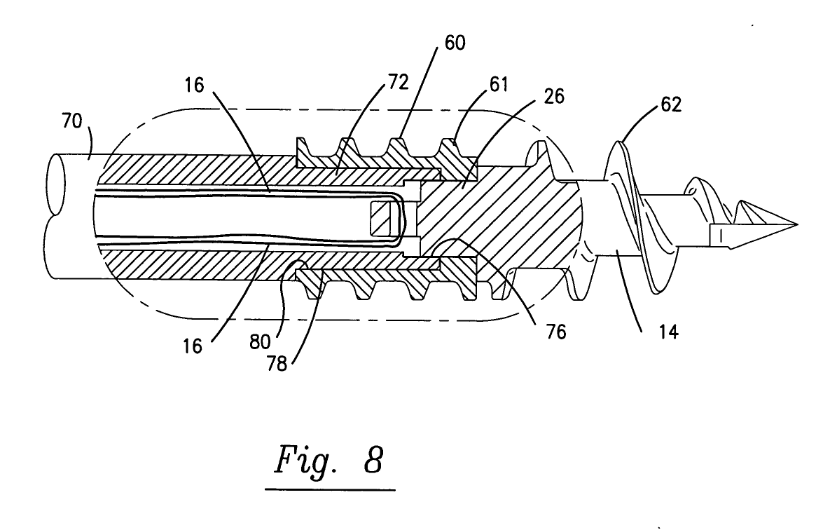

In an alternate embodiment of the invention the thread 60 on the extension 61 can have a different pitch than thread 62 on the base suture anchor 64, as shown in FIG. 8. In this embodiment the screw pitch of the extension would be optimized for cortical bone engagement whereas that of the base anchor is optimized for cancellous bone engagement.

Also shown in FIGS. 7 and 8 is a configuration in which driver 70 is provided with a driving member 72 having a non-circular drive surface 76 facing inwardly and a non-circular drive surface 78 facing outwardly. Drive surface 76 is adapted to engage drive member 26 of the base anchor 64 while drive surface 78 is adapted to engage a complementarily shaped drive surface 80 on the interior of extension 61.

The dimensions of the various components are selected based upon the degree to which the pieces are to be secured together. Also, the relative sizes of the driving and driven members may vary as a function of the materials from which the components are made. For example, the surface area of a non-metallic driven member may need to be larger than if the driven member was made of metallic material.

It will be understood by those skilled in the art that numerous improvements and modifications may be made to the preferred embodiment of the invention disclosed herein without departing from the spirit and scope thereof.

What is claimed is:

1. A suture anchor extension for use with a base suture anchor having an axis, a distal end adapted to engage a bone hole and a proximally situated drive member for driving said base suture anchor comprising:
a hollow sleeve having an axis, a proximal end, a distal end, and an outer surface;
a drive member engaging means situated at the distal end of said sleeve and adapted to engage said drive member situated at said proximal end of said base suture anchor;
a drive member situated at said proximal end of said hollow sleeve driving said hollow sleeve into the bone hole;
at least one tissue engaging projection situated on said outer surface.

2. A suture anchor extension in accordance with claim 1 wherein said drive member engaging means and said drive member are aligned along the axis of said hollow sleeve.

3. A fully threaded suture anchor comprising:
a base suture anchor comprising an axis, a body having an exterior surface and a first thread profile on said exterior surface, a drive portion proximal to said thread profile, an eyelet extending transversely through said drive portion for receiving suture;
a suture anchor extension comprising a hollow body having an axis and comprising an exterior surface, a predetermined second thread profile on said exterior surface, said second thread profile extending along the length of said sleeve, a first drive means for engaging said drive portion and a second drive means for being engaged by a driver.

4. A fully threaded suture anchor according to claim 3 wherein said first thread profile has the same pitch as, and smoothly transitions to, said second thread profile.

5. A fully threaded suture anchor in accordance with claim 3 wherein said base suture anchor and said suture anchor extension are secured together.

6. A fully threaded suture anchor in accordance with claim 3 wherein said base suture anchor and said suture anchor extension are contiguous.

7. A fully threaded suture anchor in accordance with claim 3 wherein said base suture anchor and said suture anchor extension are press-fit together.

8. A fully threaded suture anchor according to claim 4 wherein said first thread profile is adapted to be received in cancellous bone and said second thread profile is adapted to be received in cortical bone.

9. A combination comprising:
a base suture anchor comprising an elongated body having a first axis, a distal end with a bone engaging projection, a proximal end with a first non-circular drive member, an eyelet and a suture received in said eyelet;
a suture anchor extension comprising:
a hollow sleeve having a second axis in line with said first axis, a proximal end, a distal end, and an outer surface;
a non-circular drive member engaging means situated at said distal end of said hollow sleeve in engagement with said first non-circular drive member;
a non-circular driver engaging means situated at said proximal end of said hollow sleeve;
at least one bone engaging projection situated on said outer surface; and
a driver for engaging said driver engaging means at said proximal end of said suture anchor extension to drive said extension and said base suture anchor together into a work site.
